(19) United States
(12) Patent Application Publication
(10) Pub. No.: US 2023/0052603 A1
(43) Pub. Date: Feb. 16, 2023

(54) SYSTEM AND METHOD FOR DATA PROCESS

(71) Applicant: AI Clerk International Co., LTD., New Taipei City (TW)

(72) Inventor: Ru-Yng Chang, New Taipei City (TW)

(21) Appl. No.: 17/568,720

(22) Filed: Jan. 5, 2022

(30) Foreign Application Priority Data
Jul. 28, 2021 (TW) 110127614

(51) Int. Cl. G06F 16/28; G06F 16/901; G06N 5/00; G16H 10/60

(52) U.S. Cl. CPC G06F 16/287; G06F 16/288; G06F 16/9024; G06N 5/003; G16H 10/60

(57) ABSTRACT

A system for data process comprises an operating platform for storing and reading a data unit. A data processing module signally connected to the operating platform. The data unit is structured or unstructured. The data processing module labeling and processing the data unit, and generating a visualization diagram. The system for data process includes a graphical user interface, which can achieve one of the purposes of this present disclosure of improving the data visualization of structured data and unstructured data.

### CROSS REFERENCE TO RELATED APPLICATION

The present application claims priority to Taiwan Patent Application Serial No. 110127614, filed on Jul. 28, 2021, which is hereby incorporated by reference in their entirety.

### FIELD

The present disclosure relates to a system and a method for data process, more particularly to a system and a method for data process used for visualizing data.

### BACKGROUND

An unstructured data and a semi-structured data can be written freely, which can ensure that the data is more abundant and complete. However, for computers, unstructured data and semi-structured data are more difficult to be recognized than structured data. Therefore, the existing natural language processing (NLP) is to improve the computer's understanding of unstructured and semi-structured data, and further to be able to apply to the unstructured and semi-structured data.

NLP has been developed and applied quite maturely. It includes the application that can automatically extract unstructured text or semi-structured text and recognize its semantic category. This means that one of the NLP applications can transform the content of the text into semantically structured data. In this way, personnel may use the structured data for further processing, statistics, analysis and application.

NLP is more conducive to data analysis and understanding in various professional fields, and can also assist industries in making decision and judgments, deriving various benefits, such as: retail, finance, medical care . . . etc. Specifically, the application of NLP in the medical field can assist in understanding various data trends and statistical analysis of patients, diseases, etc. from various clinical records and medical literature. In addition, various analyses are more helpful to medical research, which in turn affects clinical decision-making, reduces misdiagnosis and avoids unnecessary medical treatments.

However, in the processing and application stage, because NLP needs to comprehend interdisciplinary knowledge including the knowledge of linguistics and information science with a higher threshold to learn, it is difficult for non-IT (information technology) personnel to get started. In addition, the natural language analysis tools for non-IT personnel are very rare, which hinders the progress of natural language applications to industries. Taking the medical field as an example, the application of NLP in this field is urgently to be improved due to the diverse data content and data types in the medical field. For example, the content of a “medical record of out-patient service” may be symptoms, symptom duration, symptom severity, personal disease history, etc., which usually are stored in unstructured data formats. Similarly, a “patient examination report” stored in unstructured format often records the findings/diseases, the size of the findings/diseases, the time, and so on. Conversely, the gender, date of birth, educational background, etc., often recorded in the patient's background information, are usually stored in a structured data format. Accordingly, the problem is that it is difficult for non-IT personnel to utilize the aforementioned diverse data format content in lack of available NLP tools.

In addition, NLP can assist personnel in professional fields to achieve the purpose of data analysis. However, to achieve the goal, operators who perform NLP and analysis need to be capable of making decisions with professional knowledge in the field. Also, different roles may need to perform the analysis in different aspects. For example, the data to be analyzed and observed to a physician or a pharmacist in the medical care field may be different; the data which a doctor wishes to observe or analyze is also different among different doctors. The point is that, given the gap in the background and demands of operators, the field of NLP still needs inventions that can solve the aforementioned problems.

### SUMMARY OF THE DISCLOSURE

A system for data process, comprising: an operating platform for storing and reading a data unit; a data processing module signally connected to the operating platform; the data unit is structured or unstructured; the data processing module labelling and processing the data unit, and generating a visualization diagram.

A method for data process, a data processing module is used to process one of unstructured data and structured data, to process at least one data unit, and perform the following steps: (A) a step for project creation: an operating platform generates a project data set for accessing the at least one data unit including a plurality of data values; the project data set includes at least one data attribute; (B) a step for semantic labeling decision and labeling process: the data processing module processes the at least one data unit, determines a semantic labeling decision, and outputs a labelled-up data unit, the semantic labeling decision makes one of the plurality of data values to correspond to the at least one data attribute; when the at least one data unit is unstructured data format, the data processing module performs a step for automatic semantic labeling to complete the semantic labeling decision makes one of the plurality of data values to correspond to the at least one data attribute; (C) a step for data unit storage: the data processing module stores the labelled-up data unit to the project data set; (D) a step for process and output: the data processing module processes the project data set to generate a visualization diagram.

### BRIEF DESCRIPTION OF THE DRAWINGS

FIG. 1 is a block diagram I of the first embodiment of the present disclosure.

FIG. 2 is a block diagram II of the first embodiment of the present disclosure.

FIG. 3 is a block diagram I of the second embodiment of the present disclosure.

FIG. 4 is a block diagram II of the second embodiment of the present disclosure.

FIG. 5 is a block diagram of the third embodiment of the present disclosure.

FIG. 6 is a block diagram of a visualization diagram editing interface of the third embodiment of the present disclosure.

FIG. 7 is an illustration of a data unit in the third embodiment.

FIG. 8 is a block diagram of the fourth embodiment of the present disclosure.

FIG. 9a is a variation of the fourth embodiment of the present disclosure.

FIG. 9b is an example diagram showing the result of co-occurrence analysis in the fourth embodiment of the present disclosure.

FIG. 9c is an illustration of a variation of the fourth embodiment of the present disclosure.

FIG. 10 is a block diagram of the fifth embodiment of the present disclosure.

FIG. 11 is a block diagram I of the sixth embodiment of the present disclosure.

FIG. 12 is a flowchart of a method for data labeling according to the sixth embodiment of the present disclosure.

FIG. 13a is a flowchart of a data processing method disclosed in this disclosure.

FIG. 13b is a block diagram of a system for data processing of the present disclosure.

FIG. 13c is a flowchart of another method for data process of the present disclosure.

FIG. 13d is a flowchart of another method for data process of the present disclosure.

### DETAILED DESCRIPTION

The term “signally connected” used in the detailed description of the present disclosure (including the scope of the claims) can refer to any direct or indirect connection means. For example, if the text describes that a data processing module signally connected to an operating platform, it should be interpreted as that the data processing module can be directly connected to the operating platform, or the data processing module can be connected by other devices or certain connection means, indirectly connected to the operating platform. In addition, wherever possible, elements/components/steps with the same reference numerals in the drawings and embodiments represent the same or similar parts. Elements/components/steps that use the same reference numerals or use the same terms in different embodiments may refer to related descriptions.

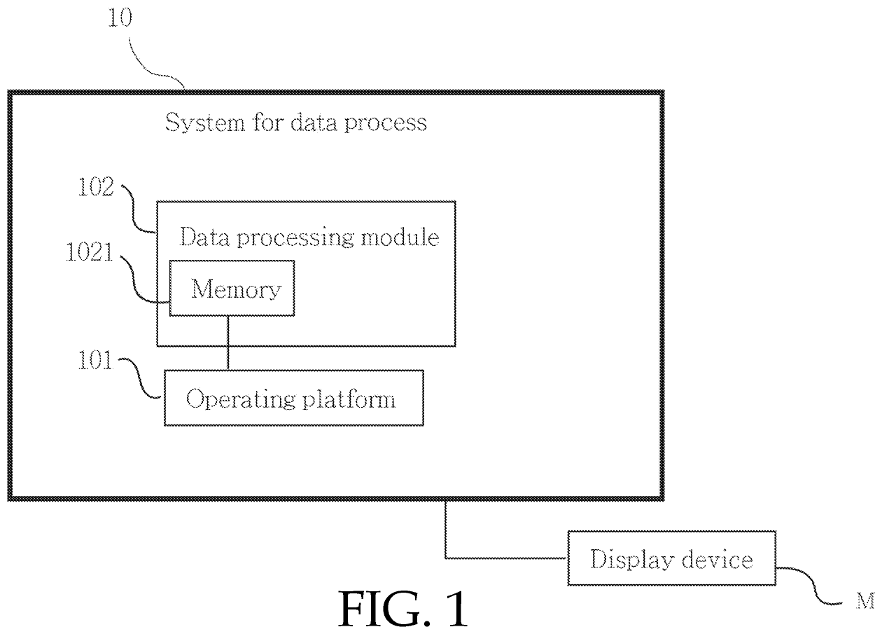

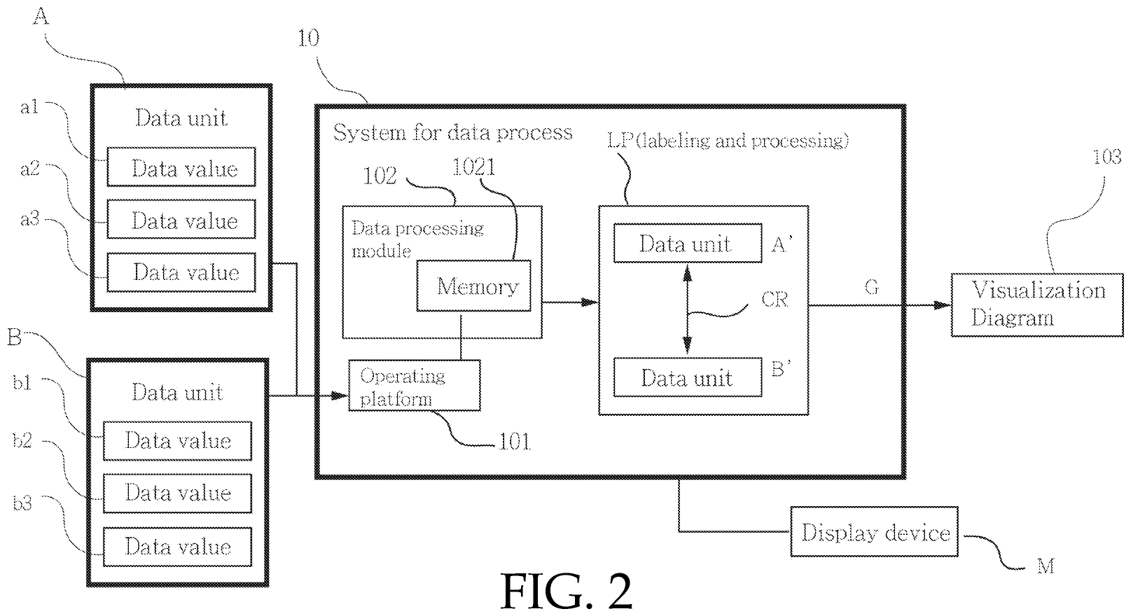

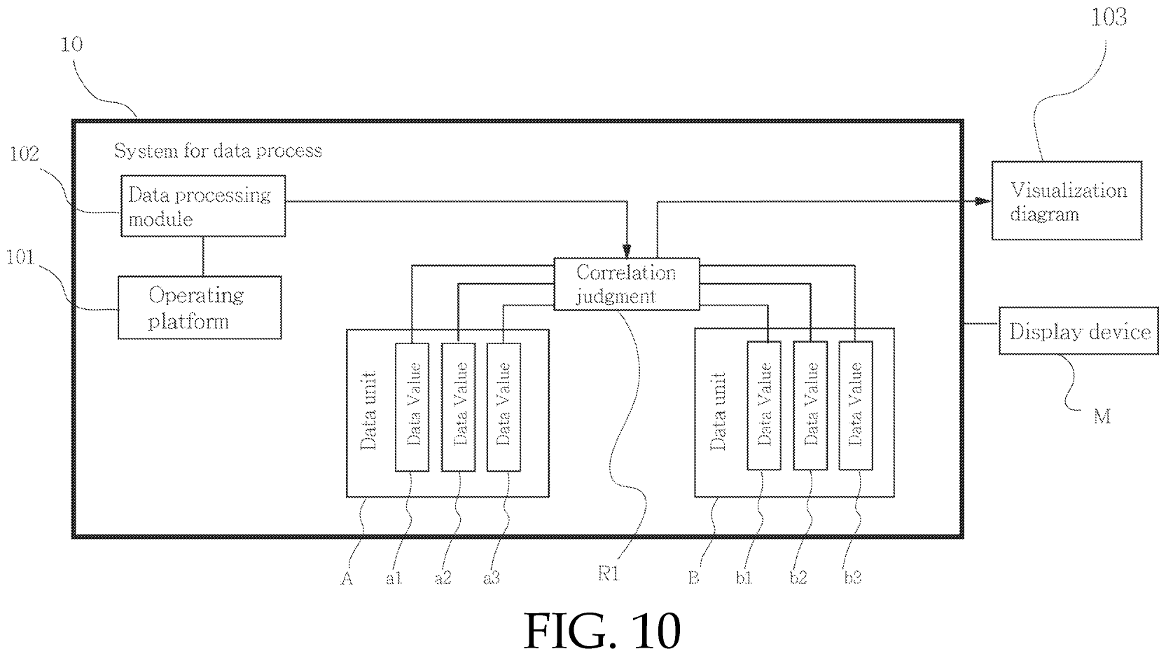

Refer to FIG. 1 to FIG. 2, which are block diagrams of the first embodiment of the present disclosure. The system for data process 10 of this embodiment can process data unit A, data unit B, or both, and generate a visualization diagram 103. The system for data process 10 mainly includes an operating platform 101 signally connected to a data processing module 102, the data processing module 102 includes a memory 1021, and the data processing system 10 is signally connected to a display device M, wherein:
(1) Data unit A and data unit B can be in a structured data format or an unstructured data format. If data unit A and data unit B are stored in a structured data format, it means that data unit A and data unit B already contain data attributes, fixed fields, specific order, or other scheme used to classify data records or data values. For example, file formats commonly used to record structured data formats such as csv, xls and other file formats. Taking the medical industry as an example, examples that often recorded in structured data formats are: patient data, medication record, drug data, medicine data (pharmaceutical raw materials), doctor's advice data, doctor's data, equipment data, department data, hospitalization data, specialty data, examination data, shift report, etc.
(2) If data unit A and data unit B are stored in unstructured data format, it means that the data unit is not stored in the form of data attributes, fixed fields, or specific order. Examples of unstructured data formats are: original is text data, including news reports, medical records, community forum posts, texts of reports, emails, etc. or other similar texts; or speech-to-text texts, including from sources of: telephone customer service, meeting recordings, or other similar texts; or even PDF files, pictures and video data with texts, including: subtitles, graphic works, or other similar data. Among them, unstructured data also refers to semi-structured data, that is, a data unit contains a part of data stored in structured data format. For example, the file formats commonly used to record semi-structured data formats are such as JSON, XML, HTML and other file formats. File formats commonly used to record unstructured data formats such as PNG, PDF and other file formats. However, the structured data format or unstructured data format referred to in the present disclosure is not limited to the previous disclosed examples. Taking the medical industry as an example, examples often recorded in unstructured data formats are: unstructured text, medical record, doctor's advice data, patient data, medication record, medicine (pharmaceutical raw materials) profile data, drug profile data, doctor's data, equipment data, and department data, hospitalization data, examination data, shift report, etc.
(3) The operating platform 101 is for a system user of the system for data process 10, and the operating platform can be presented on a display device M with a graphical user interface (GUI). The operating platform 101 is for the system user to store the data unit A, the data unit B, or both in the memory 1021; or to read the data unit A, the data unit B, or both from the memory 1021. The memory 1021 can be a scratch pad memory or a storage device such as a hard disk.
(4) The operating platform 101 is for the system user to import data unit A, data unit B, or both and store them in the memory 1021. The operating platform 101 is for the system user to give an execution command to the data processing module 102. The operating platform 101 includes a GUI for the system user to create a relationship between the data unit A and the data unit B. Specifically, it can be implemented by applying the primary key and foreign key in the concept of a relational database. The operating platform 101 provides a graphical user interface, allowing the system user to establish a key between any two of a plurality of data units by clicking.
(5) The data processing module 102 is used to “label” and “process” the data unit A, the data unit B, or both which are stored in the memory 1021, and generates a visualization diagram 103. The system for data process 10 is used for transmitting the visualization diagram 103 to the display device M for viewing by the system user.
(6) When the data processing module 102 executes “labeling”, the data processing module 102 labels the text content of the data unit A, the data unit B, or both. The labelled data unit A and data unit B are labelled as labelled data unit A′ and labelled data unit B′ in the FIG. 2. The labelled text, value, or data in the labelled data unit A′ and the labelled data unit B′ is stored in a structured data format and used as a material for generating the visualization diagram 103.
(7) This embodiment includes two “labeling” means: one is that the system for data process 10 can be used by the system user to perform manual labeling with the operating platform 101. To further illustrate, the manual labeling includes: the system user gives a labeling execution command by the operating platform 101 to make the data processing module 102 label the text content of the data units A and B. Another “labeling” mean is that the data processing module 102 can automatically label the data units A and B. To further illustrate, in order to achieve the function of automatic labeling, the data processing module 102 includes an automatic data labeling module (as an embodiment disclosed in FIG. 10) to automatically identify the text content of the data units A and B, and execute the labeling.
(8) After the data processing module 102 executes “labeling”, it “processes” the labelled data unit A′, the labelled data unit B′, or both, and generates a visualization diagram 103. The data processing module 102 may include, for example, Matplotlib, Pyecharts, Plotly, Bokeh, Seaborn, Python-based visualization plug-ins, R language-based visualization plug-ins, or other database-based visualization tools to process labelled data units A′, labelled data unit B′, or both. To further illustrate, when the data processing module 102 performs “processing”, a data correlation CR of the labelled data unit A′ and the labelled data unit B′ is created, and a visualization diagram 103 is generated based on the data correlation CR; wherein, the data correlation CR may be a relationship key or a foreign key created between the data unit A and the data unit B, which the key and the foreign key is used to create data correlation between the data unit A and the data unit B.

As aforesaid, when the data units A and B stored (or temporarily stored) in the memory 1021 of the operating platform 101 are unstructured data units, the operating platform 101 allows the system user to choose whether to perform manual labeling the data units A and B by provided GUI on the operating platform 101 or automatic labeling the data A and B by the data processing module 102. When the system user performs manual labeling, the operating platform 101 allows the system user to determine or edit a label of the data unit A and the data unit B.

This embodiment can be applied to both the data unit A and the data unit B are in a structured data format or an unstructured data format; or it can be applied to the two data units, where one of the data unit A and the data unit B is in the unstructured date format. As a result, one of the effects achieved by this embodiment: system users can easily operate the data unit A and the data unit B, which in different data formats, by the operating platform 101 to achieve the purpose of visualizing data, wherein this embodiment provides a better user experience; also, it is easy for users to use and good for introducing to various industry categories.

In another embodiment, the data unit A, the data unit B, or both may be a labelled data unit A′ or a labelled data unit B′ after performing “labeling” in advance. In this way, the previously labelled text content can be imported to the data processing module 102 by the operating platform 101 and enable the data processing module 102 to process and generate the visualization diagram 103. Thereby, the data processing module 102 can save labeling time.

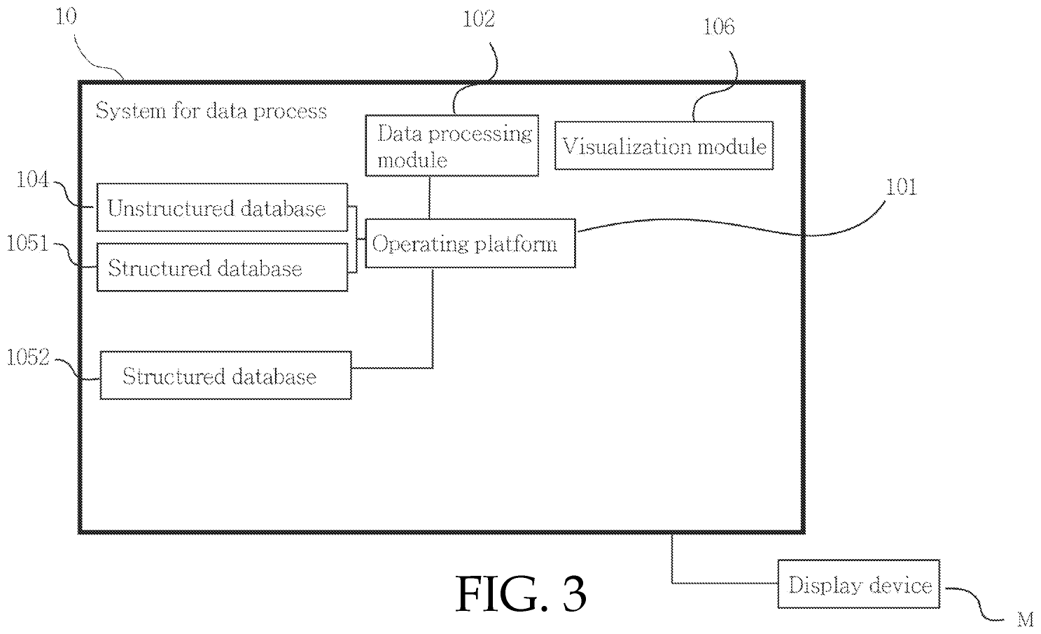

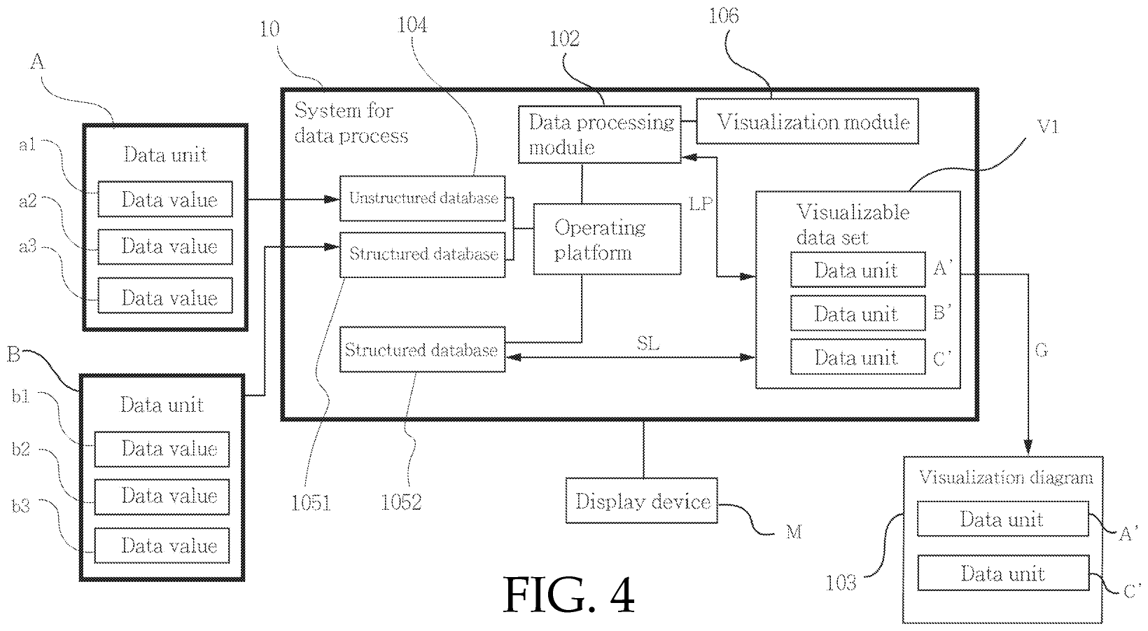

Please refer to FIGS. 3 and 4 for block diagrams of the second embodiment of the present disclosure. The system for data process 10 includes a visualization module 106, an unstructured database 104, a structured database 1051, and a structured database 1052 signally connected the operation platform 101.

The visualization module 106 signally connected the data processing module 102, may include, for example, Matplotlib, Pyecharts, Plotly, Bokeh, Seaborn, Python-based visualization plug-ins, R language-based visualization plug-ins, or other database-based visualizations tools.

The unstructured database 104 is used to store the data unit A in an unstructured data format. The structured database 1051 is used to store the data unit B in the structured data format. Specifically, the database can be a database management system similar to MySQL, MSSQL, Apache Hbase, etc.

The operating platform 101 can provide a system user to access the data unit A from the unstructured database 104 or the data unit B from the structured database 105.

The data processing module 102 is used to process the data unit A and the data unit B, and can output a visualizable data set V1. The system user can even import the visualizable data set V1 to the data processing module 102 by the operating platform 101. The visualization module 102 generates a visualization diagram 103 according to the visualization data set V1.

The visualizable data set V1 includes one or more than one data unit that is able to be visualized by the data processing module 102, for example, data units in a structured data format. In this embodiment, the visualizable data set V1 includes a labelled data unit C′, which is labelled by the data processing module 102 in advance. The data processing module 102 can copy or move the labelled data unit A′ or the labelled data unit B′ to the visualizable data set V1.

The system user can use the operating platform 101 to enable the data processing module 102 to process the labelled data unit A′, the labelled data unit B′, the labelled data unit C′, or a combination thereof in the visualizable data set V1. Thereby, when the system user operates the operating platform 101, the data processing module 102 can read one or more than one of the plurality of data units in the visualizable data set V1, and generate a visualization diagram 103. To achieve aforesaid purpose, the visualizable data set V1 can be stored in the structured database 1052. The data processing module 102 processes the labelled data unit A′ and the labelled data unit C′ in the visualizable data set V1 to generate the visualization diagram 103.

The structured database 1052 of this second embodiment can be used to store and read the visualization data set V1. In this way, the data units that have been labelled in advance or the data units that are labelled and processed LP can be stored in the structured database 1052 by the visualizable data set V1. The operating platform 101 can read and import the visualizable data set V1 to the data processing module 102 by the structured database 1052, in order to output the visualization diagram 103. In this way, the operations of a system user can be simplified, and the processing time required by the system can be shortened. In another variation of the second embodiment, the data unit that has been labelled or the data unit that has been labelled and processed LP can be stored as a computer-readable file, such as JSON; it is not a requirement to be stored in a structured database or unstructured the database.

In another variation of the second embodiment, the structured database 1051 and the structured database 1052 may be the same one.

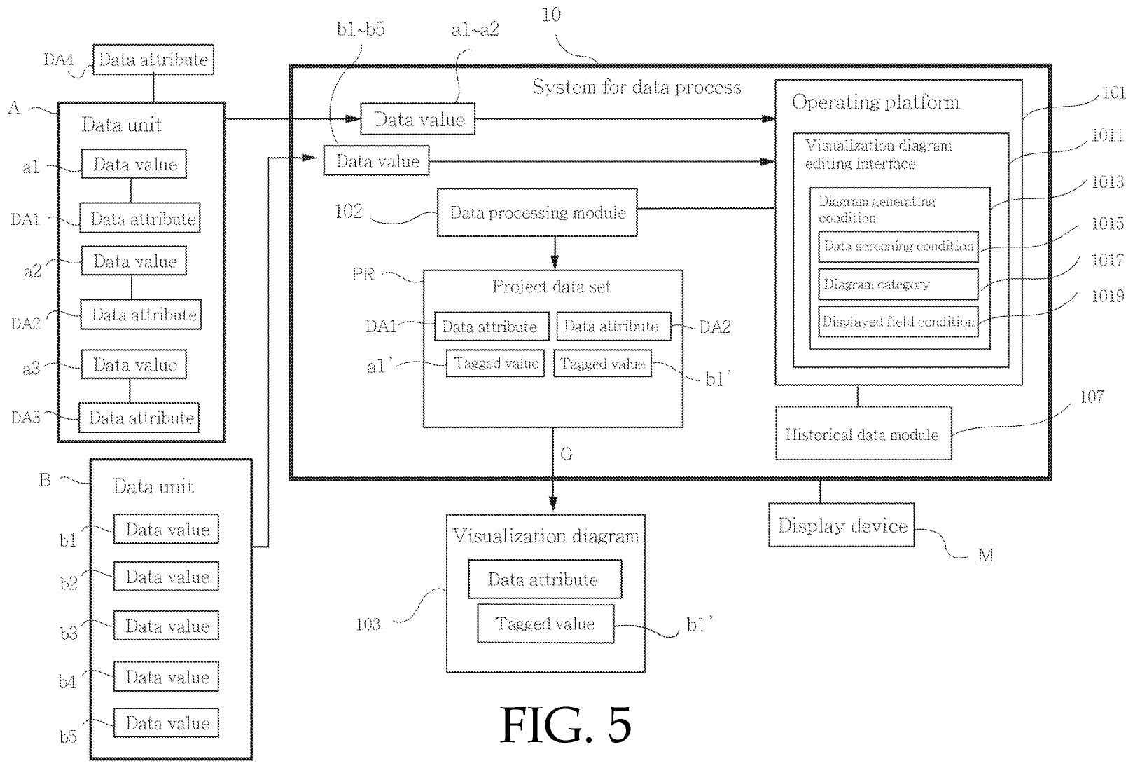

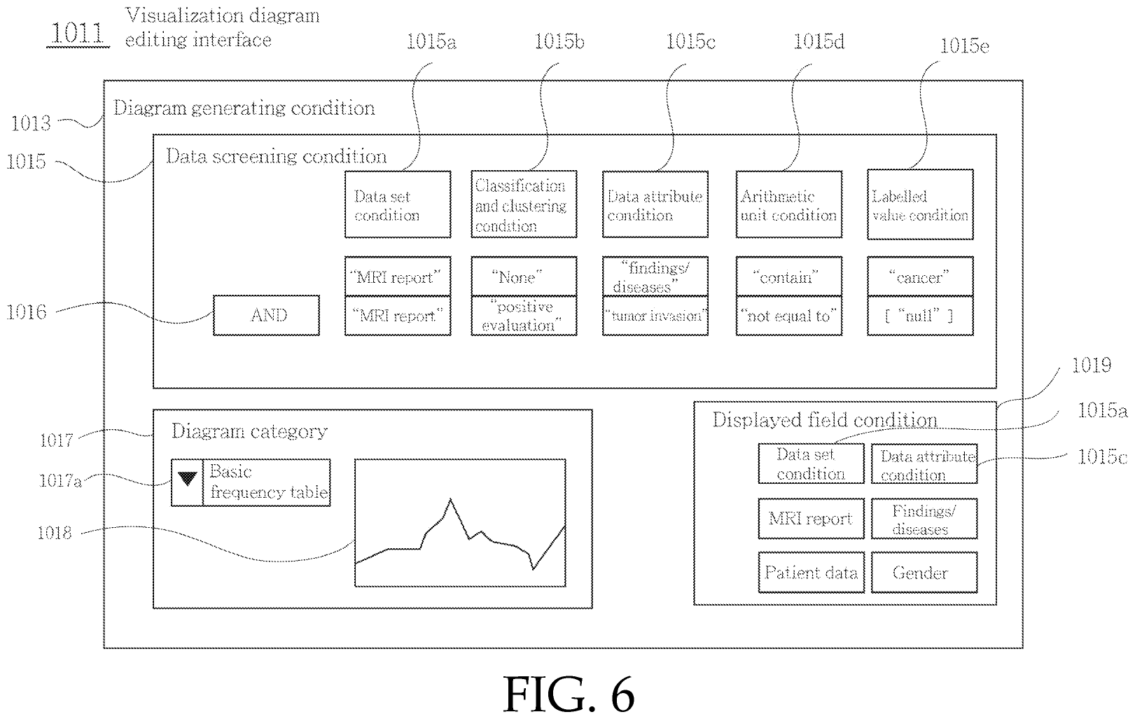

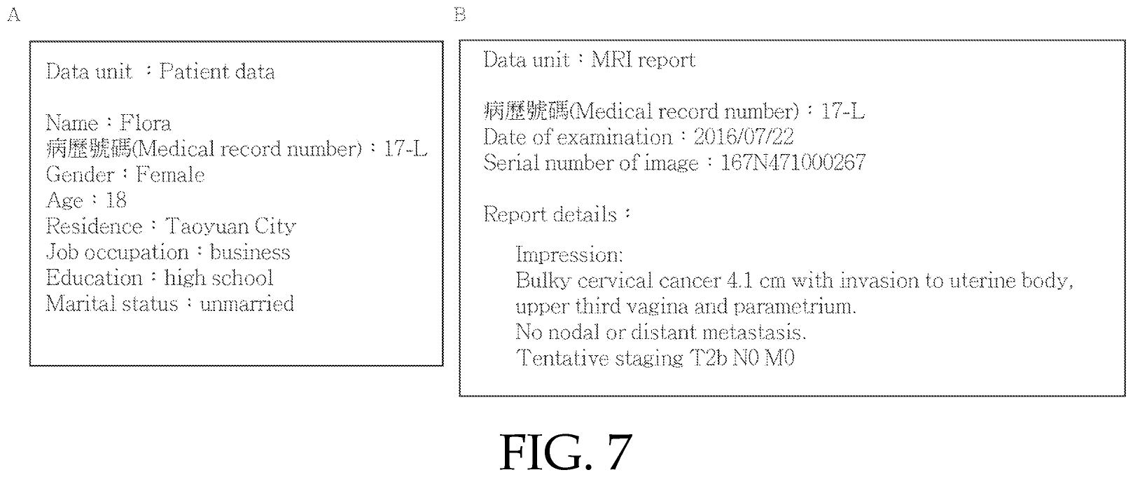

Next, please refer to FIG. 5 for a block diagram of the third embodiment of the present disclosure. FIG. 6 is a block diagram of a visualization diagram editing interface of the third embodiment of the present disclosure. FIG. 7 is an illustration of a data unit in the third embodiment. The operating platform 101 may include a visualization diagram editing interface 1011, which is used for setting a generation condition of the visualization diagram 103. The system for data process 10 includes a historical data module 107 signally connected to an operating platform 101 and a data processing module 102. The system user can operate the operating platform 101 to generates a project data set PR. The operating platform 101 can edit and revise the project data set PR. The project data set PR can be temporarily stored or stored in the system for data process 10.

Data Value

In the third embodiment, the data unit A is a structured data format, such as patient data. The data unit B is an unstructured data format, such as an MRI report. The data unit A includes data values a1 to a3, and the data unit B includes data values b1 to b5.

The data values a1 to a3 can be stored in the data unit A in a structured data format. Specifically, the data value refers to a value stored in a storage field in a data unit, such as a character, a word, a value, a bit value, etc. For example, if the data unit is patient data in a structured data format, the data value can be a record or a tuple in the data unit, such as “name,” “Flora(name),” “ [symbol] (Medical record number),” “17-L.” In this case, the data value may include multiple characters, numbers, bit values, or symbols. In other embodiments of the present disclosure, the data value after being labelled by the data processing module 102 may be the aforementioned record or tuple including multiple characters, numbers, bit values, or symbols.

The data values b1 to b5 refer to a character, a number, a bit value, or a symbol in the data unit B that can be recognized by the data processing module 102, for example, Arabic numerals, Chinese characters, English characters, or symbols. For further example, if the data unit is a medical record table in an unstructured data format, the data values can be, for example: “n,” “a,” “m,” “e,” “f,” “l,” “o,” “r,” “a,” “ [symbol] ,” “ [symbol] ,” “ [symbol] ,” “ [symbol] ,” “1,” “7,” “-,” “L,” “c,” “a,” “n,” “c,” “e,” “r.” The labelled value a1′ and the labelled value b1′ refer to the words, values, or data of the data unit A and the data unit B after being automatically or manually labelled by the data processing module 102, for example: “flora,” “17-L,” “cancer.”

Data Attributes

The data attributes DA1 to DA3 can be “field name” and “label name” in a structured data format. More specifically, for example: “medical record number,” “name,” “gender,” “residence,” “time,” “the findings/diseases,” and “tumor invasion location” in order are the data attributes of the data “17-L,” “Xiao-Ming(name),” “Male,” “Taoyuan City,” “Jul. 15, 2021 at 10:10 AM,” and “Liver Cancer,” “Liver.”

In some embodiments, the operating platform 101 can import the data value a1 or the data value b1 of the data unit A or the data unit B to the data processing module 102. The system user can manually label the data value a1 corresponding to a data attribute DA1 through the operating platform 101, and then store in the project data set as the data value a1′. For example, the data unit A in the structured data format includes data values: “job occupation”, “office worker”. Use symbols such as “#”, “$”, “%”, or other characters to label before the data value a1, for example: “#job occupation”. In this way, the data processing module 102 can identify “job occupation” as a data attribute.

Specifically, in some other embodiments, the operating platform 101 may further define a data attribute as a semantic labeling decision. That is, a data attribute tag is a “semantic tag” in the natural language processing. After labeling up some unstructured data formats, the unstructured data formats can be labeled with semantic tags through the data processing module.

In some embodiments, the data attribute can further provide the data processing module to be used for statistical analysis of clustering or classification. For example, the data attributes can be “residence”, “marital status”, “education”, “age”, etc., which are usually used as data attributes for clustering or classification in statistical analysis. In this way, the visualization diagram can present a diagram showing the classifications or the clusterings.

In some embodiments, as shown in FIG. 5, the data values a1 to a3 stored in the data unit A may already be corresponded respectively to the data attributes DA1 to DA3. Also, in the project data set PR, it may have the same data attribute DA1 and data attribute DA2.

In some embodiments, the data attributes may be used by the system for data processing as a basis for classification (for clustering or classification). For example, the data attribute may be “positive comment”, “symptoms”, “unpopular”, “region”, or other similar classifications or clusterings. In this way, the visualization diagram can present a diagram of classification or clustering. In other embodiments, a clustering or a classification may further include one or more clusterings or classifications. That is, the clustering or the classification can include one or more superordinate categories, and the one or more superordinate categories include one or more sub-categories. For example, the superordinate category may be “Northern of Taiwan”; the sub-categories may be “Taipei City”, “New Taipei City”, etc. To further explain, that is, each data attribute can include one or more data attributes.

Project Data Set
The operating platform 101 can edit the project data set PR. Specifically, the project data set PR allows a system user to establish the correct data attribute corresponding to the data unit through the operating platform 101. The project data set PR can create data attributes DA1 and DA2 in advance; or based on the data attribute DA1 and data attribute DA2 provided in the imported data unit A, it is automatically created in the project data set PR; alternatively, create the corresponding data attribute DA2 to the project data set PR according to the data attribute which is determined by the data processing module 102 after it labeling the data unit B. For example:
The data attribute DA1 is “medical record number.”
The data attribute DA2 is “discovery.”
Data attribute DA3 is “age.”
The data value a1 is “17-L.”
The data value a2 is “cervix cancer.”
The data value a3 is “36”.
The data values b1-b5 are “m”, “y”, “o”, “m”, and “a” respectively. The data processing module 102 labels the data values b1-b5 as “the findings/diseases.”
The labelled value a1′ is “17-L”. The labelled value b1′ is “myoma”, and the data processing module 102 can store the labelled value b1′ of the “the finding/diseases” in the project data set PR corresponding to the data attribute DA2.

In some embodiments, the data processing module 102 can identify that the data unit A or the data unit B is an unstructured data format or a structured data format.

Then please refer to FIG. 6 and FIG. 5. The visualization diagram editing interface 1011 can determine a chart generating condition 1013; the diagram generating condition 1013 includes data screening condition 1015, diagram category 1017, and displayed field condition 1019. The data processing module 102 can process the labelled value a1′ and the labelled value b1′ according to the diagram generating condition 1013 determined by the system user, and generate the visualization diagram 103. The operating platform 101 transmits the determined diagram generating condition 1013 to the data processing module 102 for processing, and generates a visualization diagram 103. Each feature is described as follows:
(1) Diagram generating condition 1013:
   The diagram generating condition provide a graphical user interface for the system users. The diagram generating condition 1013 may include different types of conditions, which are able to determine the processing of the labelled value a1′ and the labelled value b1′, thereby providing a desirable diagram to the user. For example, in this embodiment, the diagram generating condition 1013 includes: a data screening condition 1015, a diagram category 1017, and a displayed field condition 1019. Overall, the user can obtain the visualization diagram 103 through simple operations of the system for data process.
(2) Data screening criteria 1015:
   The data screening condition 1015 may further include: a data set condition 1015a, a classification and clustering condition 1015b, a data attribute condition 1015c, an arithmetic unit condition 1015d, a labelled value condition 1015e, or a combination thereof. The data screening condition and other conditions (1015a-1015e) shown in FIG. 6 can be arranged in appropriate positions in a graphical user interface. In this embodiment, the conditions are presented in rows, and the rows corresponding to fields below are available for system users to fill-in or selection. The aforementioned fill-in may refer to input key word through the operating platform with a keyboard; and the selection may refer to the selection of the system user with a pull-down menu.
   The system user can edit the data screening condition 1015 through the operating platform 101 and then transmit the data screening condition 1015 to the data processing module 102. The data processing module 102 can screen out the data contents required for generating the visualization diagram 103 according to the conditions (1015a-1015e) of the data screening condition 1015. As shown in FIG. 6, the data screening condition 1015 may include a group (or row), and each group (or row) of data screening condition 1015 may include one or more combination of conditions (1015a-1015e). In addition, another group (row) of data screening condition 1015 can also be added. The system user can edit the relationship between each one group of data screening condition 1015 and the other through the arithmetic unit 1016, for example: “and”, “or” and other arithmetic units.
(3) Data set condition 1015a:
   Please refer to FIG. 5 and FIG. 6. In this embodiment, due to the project data set PR is named as “MRI report,” the field below the data set condition 1015a is “MRI report.” In this way, system users may filter data they need. It is worth noting that, in some embodiments, there may be one or more project data sets PR. The system user can filter the project data set PR to be processed by the data processing module 102 according to the data set condition 1015a. In addition, based on this, the data unit A can be one of the project data sets PR. In other words, because the data unit is a structured data format, and each data value a1-a3 has the corresponding data attribute DA1-DA3. The operating platform 101 can establish a relationship key (Key) between the project data sets on the basis of the data attributes DA1-DA3. In this way, a relationship can be established between the data unit A and the project data set PR. The data processing module 102 can access the corresponding one or more data values according to the relationship key.
(4) Classification and clustering condition 1015b:
   The data attribute DA4 can be a classification and clustering condition 1015b, for example: positive reviews, good reviews, or other customizable classification. The operating platform 101 can label the data attribute DA4 of the data unit A. In some embodiments, the data processing module 102 can automatically identify the data attribute DA4 of the data unit. Specifically, a data unit can be classified by data co-occurrence analysis. For example: if there are positive semantic data such as “good”, “healthy,” etc. in a data unit at the same time, the data unit can be labelled as a “positive evaluation” clustering.
   In some embodiments, the data can be screened by the classification and clustering conditions or data attribute conditions. In this way, the system user can select the classification or the clustering to be presented in the visualization diagram by operating the operating platform.
   In other embodiments, the presentation of classifications or clustering can disclose the distribution information or summary information of different patients. Specifically, the distribution information may disclose gender ratio, area ratio, or other similar distribution information, for example, male: 40% and female: 60%. The system users can further understand the summary information of one or more “male” patients by clicking “male”. It is further explained that if a point presented on the visualization diagram represents a patient, the system user, by clicking on the point, can see the summary information of the patient.
   For instance, please refer to the content revealed by the data filter condition 1015 in FIG. 6, and adjust the conditions slightly, which can be applied to the following situation: a physician would like to find [“MRI report”] [data attribute condition (in this example, the data attribute condition is “label name”) equal to “findings/diseases”] [“contain”] [“cancer”] [“and”] [“MRI report”] [“data attribute condition (in this example, the data attribute condition is “label name”) equal to “tumor invasion”] [“not equal to”] [“null”]. Also, the bottom left diagram category 1017, the physician selects the basic frequency table. In this way, after the visualization diagram is generated, the physician can see the “basic frequency table” and analyze it in the data set MRI report, and also know which organs cancer frequently invades (tumor invasion).
   Furthermore, to be more specific, if the data is sufficient, a part of the visualization diagram will include the location of the disclosure information of “‘certain cancer’ tumor invading ‘certain organ tissue.’” Wherein in the position of the disclosed information (not shown in the figures), you can click on “‘a cancer” tumor invades ‘a certain organ tissue’ and see the related “clustering distribution” such as gender distribution, etc. The, the system user can click on a specific range of age, for example: “20-30 years old,” and see the “patient list” or “patient summary information” to learn the medical history or information of one or more patients.
(5) Data attribute condition 1015c:
   The system operator can select the data attribute condition 1015c to filter the data values that needs to be processed for visualization by the data processing module 102. As shown in FIG. 6, for example, in the field below the data attribute condition 1015c, the selection of “findings/diseases” means to filter the one or more data values, which its data attribute as “findings/diseases” in the project data set PR named “MRI report.” In this way, the one or more data values are used as the materials for generating the visualization diagram 103.
(6) Diagram category 1017:
   The diagram generating condition 1013 includes a diagram category 1017.
   The diagram category includes data list, data table, basic frequency table, percentage table, co-occurrence matrix, co-occurrence list, clustering distribution, bar chart, broken line chart, table, pie chart, histogram, statistical chart, scatter chart, bubble chart, surface chart, radar chart, horizontal bar chart, timeline chart, chart of organ and body tissue, or a combination thereof. The front-end chart types also include other similar charts that can present analysis results. The so-called chart of organ and body tissue specifically refers to the data attributes displayed in the visualization diagram including at least one of “organ” or “body tissue,” for example, “liver,” “subcutaneous tissue,” and so on.
   In some embodiments, if one or more data values corresponding to a data attribute are numerical values, the data processing module can display a visualization diagram that includes the numerical values and time in the axes. For example, the visualization diagram can display the time interval in which the data values are concentrated, or the trend of the data values in the time sequence.
(7) Displayed field condition 1019:
   The system user can select the data set condition 1015a and data attribute condition 1015c to be displayed in the visualization diagram 103. To further illustrate, the difference between the displayed field condition 1019 and the data screening condition 1015 is that the data screening condition 1015 is for the system user to filter data through the operating platform. However, the visualized diagram 103 generated each time the system user can select different displayed field conditions 1019 according to different scenarios. For example, please refer to the displayed field condition 1019 in FIG. 6. In this embodiment, both “MRI report” and “patient data” are the project data set PR. After selecting “MRI report” in the field below the data set condition 1015a, the system user can select the data attribute condition 1015c. The same to the data screening condition 1015, a group (row) of displayed field conditions 1019 can be added, and after selecting “patient data” in the data set condition 1015a, the data attribute condition, “gender,” can be selected.

The historical data module 107 is used to store the diagram generating condition 1013, the visualization diagram 103, or a combination thereof. In detail, the historical data module 107 can store the data screening condition 1015, diagram category 1017, and displayed field condition 1019 determined by the system user to the historical data module 107.

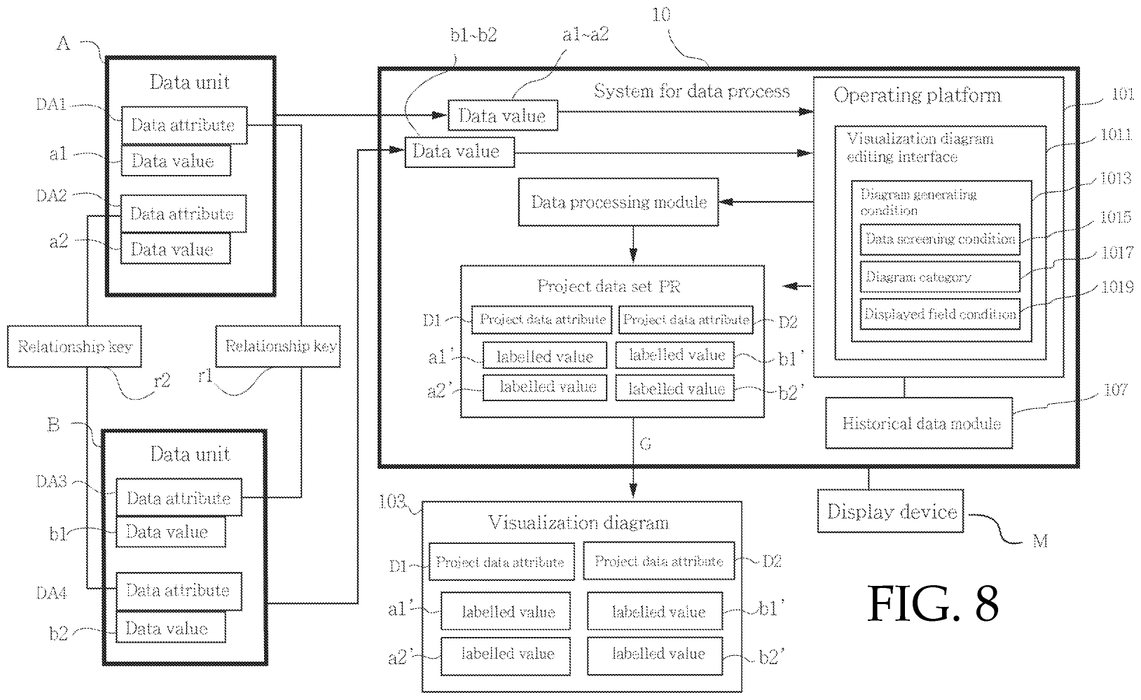

Next please refer to FIG. 8 for a block diagram of the fourth embodiment of the present disclosure. The main difference from the third embodiment is that the project data set PR in this embodiment includes project data attributes D1-D2. The operating platform 101 is further used to determine the relationship key r1-r2; the relationship key r1 corresponds to the data unit A and the data unit B. To further illustrate, the relationship key r1 corresponds to the data attribute DA1 and the data attribute DA3; the relationship key r2 corresponds to the data attribute DA2 and the data attribute DA4. In this way, the data processing module 102 can generate the visualization diagram 103 according to the relationship key r1 and the relationship key r2. To further illustrate, before the data processing module 102 processes the data values a1-a2 and the data values b1-b2 and generates the visualization diagram 103, the data unit A and the data unit B can be established a relationship by the operating platform 101; the so-called relationship can refer to the use of primary key in the relational database to establish the two, so that the data processing module can access the data values a1-a2 and b1-b2. The project data attributes D1-D2 of the project data set PR can be edited by the operating platform 101, and then the visualization diagram 103 to be generated can be set by the visualization diagram editing interface 1011. The content displayed in the visualization diagram 103 is mainly based on the project data attributes D1-D2.

In addition, the present embodiment differs from others in that both data unit A and data unit B are in a structured data format; or, data unit B has been labelled by the data processing module 102 and can be identified as a structured data format. In other words, the data value b1 and the data value b2 of the data unit B are corresponding to fields or data attributes.

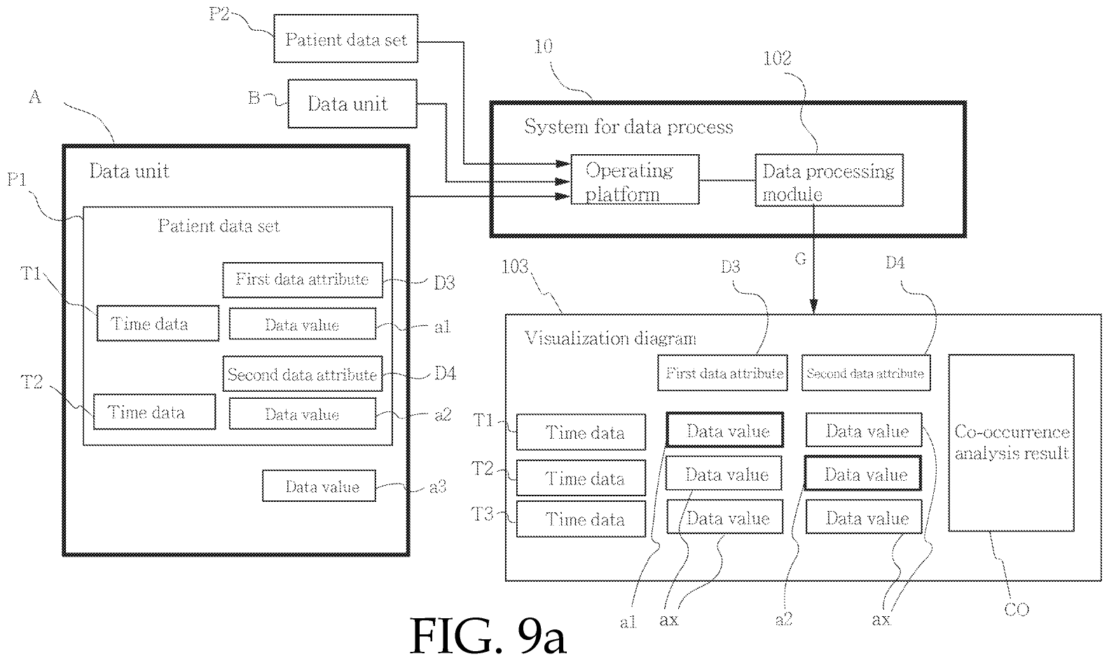

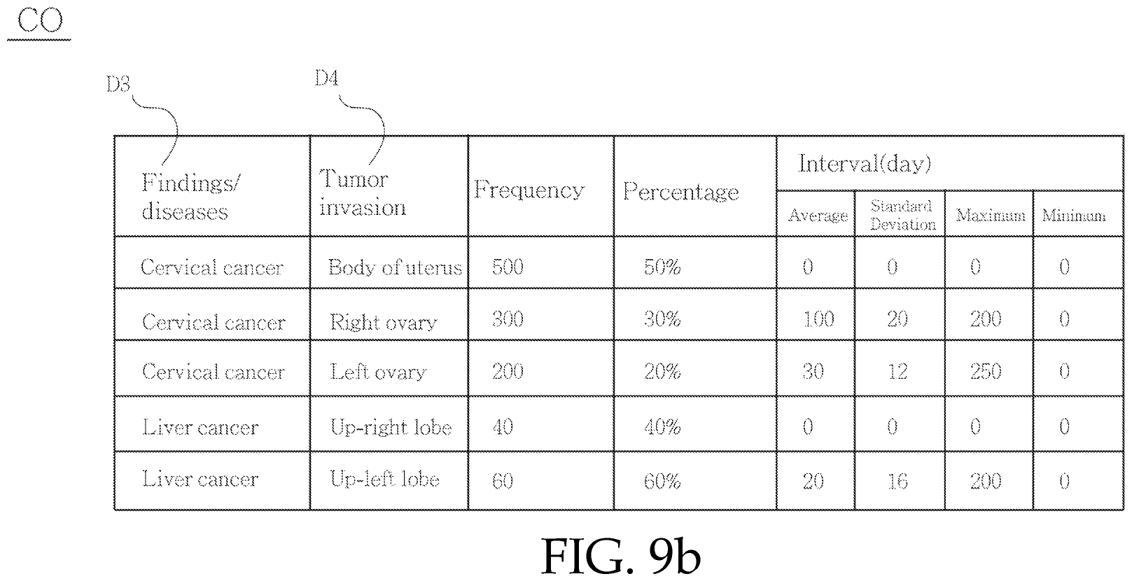

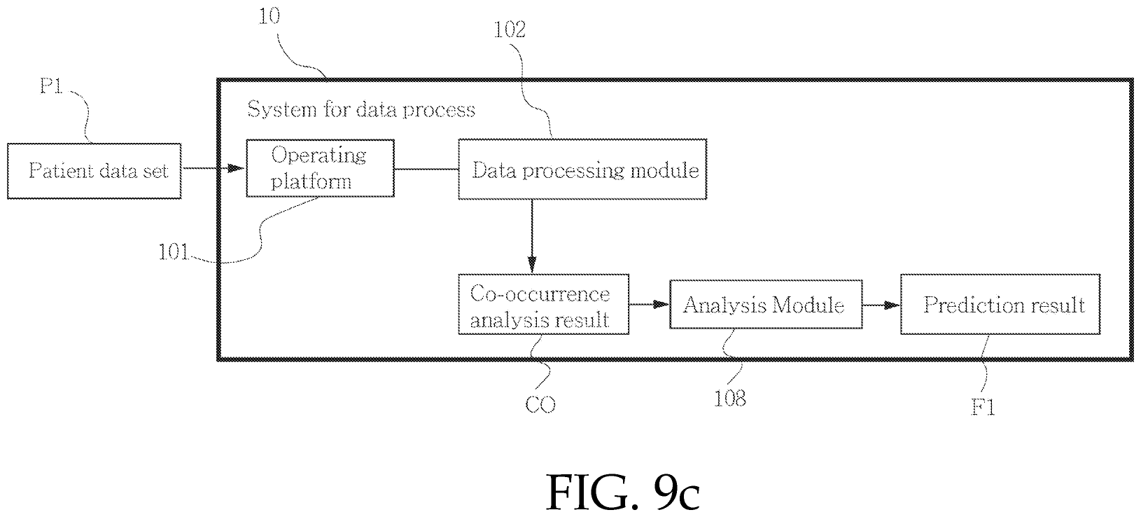

Next, please refer to FIG. 9a for a variation of the fourth embodiment of the present disclosure. The difference from the fourth embodiment is that the data unit A includes a patient data set P1. The patient data set P1 is mainly stored in a structured data format. In some embodiments, the data unit A may include a data value a3 in an unstructured data format. Similarly, the patient data set P1 can be labelled by the data processing module 102 and be generated to a visualization diagram 103, wherein a patient data set P1 can store a plural of data values a1-a2 which are respectively corresponding to a time data T1-T2. The data values a1-a2 respectively correspond to the first data attribute D3 and the second data attribute D4.

Wherein, the patient data set P1 includes data values a1-a2. The data value a1 corresponds to the first data attribute D3, and the data value a2 corresponds to the second data attribute D4. The data value a1 and the data value a2 respectively corresponds to the time data T1 and the time data T2. Time data T1-T2 can refer to the time recorded by data values a1-a2. For example, the data value a1 is “cervical cancer,” the time data T1 is “May 11, 2021,” and the first data attribute is “findings/diseases.”

In this variation, the patient data set P1 can be used to analyze the condition of a patient at different points in time. For example, a patient's “findings/diseases” continue to appear “cervical cancer”, and a certain day “Uterine Fibroids” has been added to “findings/diseases.” The above purpose can be accomplished by this embodiment, and please refer to FIG. 9a. A data unit A includes a patient data set P1 as the records of a patient's condition at different points in time. The first data attribute D3 and the second data attribute D4 are labelled as “findings/diseases” by the data processing module 102. (That is, in this embodiment, the first data attribute D3 and the second data attribute D4 can be the same or different).

The data value a1 corresponding to the first data attribute D3 is labelled as “cervical cancer.” The time data T1 corresponding to the first data attribute D3 is “May 11, 2020.” The data value a2 corresponding to the second data attribute D4 is labelled as “Uterine Fibroids,” and the time data T2 corresponding to the second data attribute D4 is “Dec. 11, 2020.” In this way, when the data processing module 102 labels and processes the data unit A, a visualization diagram 103 can be generated. Please refer to the visualization diagram 103 in FIG. 9a, which contains the time data T1 corresponding to the data value a1; the time data T2 corresponding to the data value a2. In this embodiment, for ease of understanding, the correspondences between data attributes, time data, and data values are simply presented in a table-like manner. In other embodiments, the visualization diagram may be presented in other ways, for example, the aforementioned analytic graphs such as broken line chart, pie chart, and so on.

Another example is the following scenario: a patient's “findings/diseases” is “Uterine Fibroids,” and the change in “size” of the uterine fibroids is observed during a continuous observation. Similarly, as in the mentioned embodiments, the first data attribute D3 and the second data attribute D4 of the patient data set P1 are “findings/diseases.” The data values a1-a2 are numerical data. Data values a1-a2 correspond to data time T1-T2 respectively. When the data processing module 102 labels the data value, it can be identified as numerical data.

In order to make the data processing module 102 to identify a numerical data, the data values a1-a2 may be manually labelled by the operating platform 101, or may be automatically labelled by the data processing module 102. To further explain, one may label at the front of the data value a1 by using symbols such as “#,” “$,” “%,” or other characters, for example: “#job occupation.” In this way, the data processing module 102 can identify “job occupation” as a data attribute.

Please refer to FIG. 9a. The visualization diagram 103 not only discloses the data values a1 and the data value a2, which are taken from the patient data set P1, but also other data values ax, wherein the data value ax may come from another patient data set P2 or another data unit B of the same patient. In order for the patient data set P2 and the data unit B to correctly correspond to the data attributes, the data unit A, the data unit B, the patient data set P1, and the patient data set P2 can be associated to each other by using the relationship key.

Please refer to FIG. 9a and FIG. 9b again. FIG. 9b is an example diagram showing the result of co-occurrence analysis in the fourth embodiment of the present disclosure. The visualization diagram 103 includes a co-occurrence analysis result CO. Perform co-occurrence analysis of the patient data set P1, the system user can know the implicit meaning of the data values. For example, the body organs often being invaded by specific cancers: cervical cancer and body of uterus often correspond to the same time data at the same time, implying that “cervical cancer” often invades “body of uterus.” In addition, it is worth noting that in the previous example, the time data can also provide the system user to understand the time taken for the “cervical cancer” to invade the “body of uterus.” A physician can use the co-occurrence analysis result CO to determine the body parts that need attention during surgery or examination. In addition, the physician can infer whether there is an invasion of malignant tumor, and whether to remove other body parts other than the target object in advance during the operation.

The so-called co-occurrence analysis may include analyzing data units using Pearson Product-Moment Correlation Coefficient (PPMCC); in some embodiments, Mutual Information (MI) analysis may also be applied to the data unit.

Please refer to FIG. 9b again. The co-occurrence analysis result CO may include a statistical analysis result, a probability analysis result, or a combination thereof. It is supplemented that the time data can be used to record the time of the patient's examination, the time of the patient's examination report being prepared, the time of the patient visit the hospital, the time of the patient being hospitalized, the time of the patient leave the hospital, the time of the patient's surgery start, the time of the patient's surgery end, the time of patient's birth, the time of the patient was diagnosed, the time of the patient's death, the time of a data being stored, the time of a duration, the time of the occurrence of a condition (disease/complication/symptom), the time of the end of a condition (disease/complication/symptom), or a combination thereof.

To add further supplemented explanation, by establishing a patient data set or establishing a relationship key; by establishing the relationship key, specifically, one or more data values of multiple data units can be corresponding to the same patient (corresponding to the same primary key). In this way, a particular patient can be analyzed. For instance, if one or more data values are presented on the visualization diagram as a kind of broken line graph, the point of the end point of the broken line graph can represent a data value in a patient data set P1. The system user can learn the data values, the summary or information of the data attributes stored in other data units of the patient by clicking on the point of the end point.

Please refer to FIG. 9c is an illustration of a variation of the fourth embodiment of the present disclosure. The system for data process 10 may include an analysis module 108 signally connected to the operating platform 101; the analysis module 108 is used to process the co-occurrence analysis result CO, predict the patient data set, and generate a prediction result F1. The analysis module 108 can analyze the characteristics of data values such as density, the small-world phenomenon, degree distribution, and degree correlation. The prediction result F1 may be an evaluation to the co-occurrence analysis result CO. Specifically, the prediction result F1 can be presented in a percentage, table, or other visualization approaches. In addition, the prediction result F1 can also be described in text. For instance, please refer to the co-occurrence analysis result CO shown in FIG. 9b. When a patient is diagnosed with “cervical cancer,” and the “upper right liver” and the “upper left liver” may be the location where the tumor might invade, a doctor should pay close attention to it. The special feature of this presented disclosure is that the patient data in an unstructured data format is enormous. Through this disclosed variation of the embodiment, corpus and numerical data can be quickly identified and collected, and further can be applied. In this way, we can further understand, in the results of co-occurrence analysis, whether the range of organs or body tissues and the range of time of the“tumor invasion,” which are often co-occurred along with the “cervical cancer,” are similar to the characteristics of other cancers.

Please refer to FIG. 10 is a block diagram of the fifth embodiment of the present disclosure. The difference between this embodiment and the embodiment disclosed in FIG. 2 is that the data processing module 102 can determine a correlation judgement R1. The data processing module 102 can further generate the visualization diagram 103 according to the correlation judgment R1. The data unit A and the data unit B includes a plural of data values a1-a3 and a plural of data values b1-b3. The data processing module 102 judges the data value a1 included in the data unit A and the data value b1 included in the data unit B a correlation judgment R1; or, the data value a1 and the data value b2, or the data value a1 and the data value b3. Similarly, the correlation judgment can also be any two of the data values a1-a3 included in the data unit A. Correlation judgment R1 includes: “greater than,” “equal to,” “less than,” “not equal to,” “greater than or equal to,” “including,” “less than or equal to,” or other similar logical judgement operators. The correlation judgment R1 can be set by the operating platform to determine the judgment conditions, for example: “greater than the value 0,” “less than or equal to the value 2.” The data processing module 102 generates a visualization diagram 103 further according to the correlation judgment R1. For example, if the data value is time data, the time sequence can be judged. If the data value is numerical data, the size can be judged and sorted. The content disclosed above can also be displayed on the visualization diagram 103.

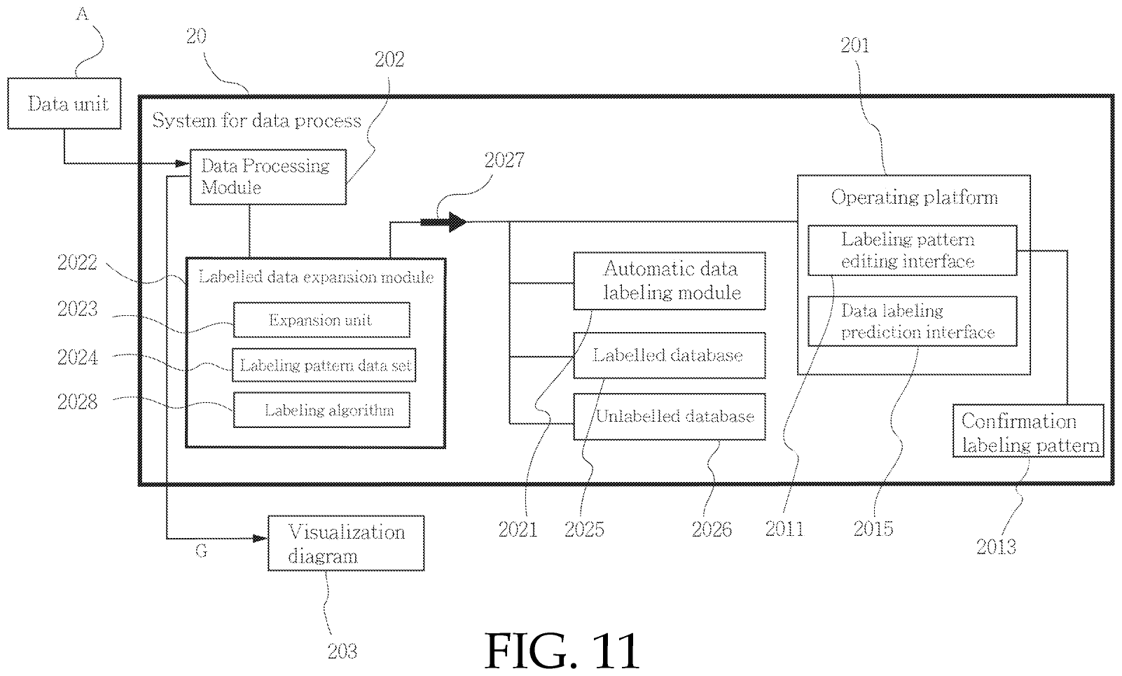

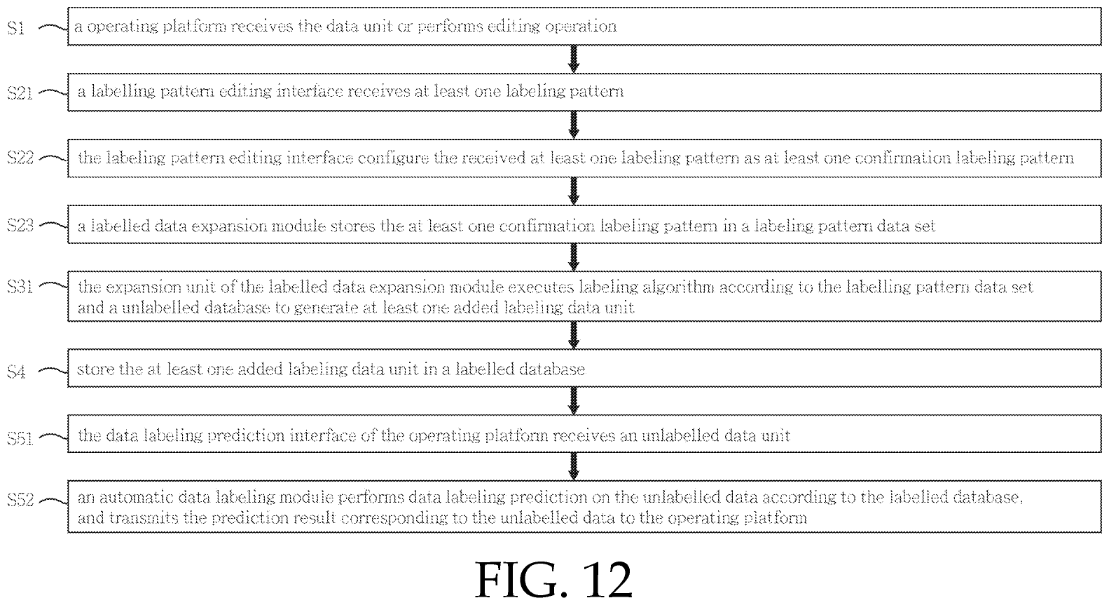

Next please refer to FIG. 10 and the sixth embodiment disclosed in FIGS. 11 and 12 described later. This present disclosure can also predict the labeling result of the data processing module. That is, when the data value is a word, numerical data, or other characters, the present disclosure can determine the data value with similar semantic meaning and correspond to the correct data attribute. Specifically, for example, the wording difference between “cervical cancer” and “cervikal cancer” can be judged by the prediction result and the correct data attribute can be labelled as “findings/diseases.”

The correlation judgment may further include logical operation. Specifically, for instance, the labelled data values of data unit A and data unit B are all numerical values, which can determine whether the data values to be used to generate the visualization diagram are redundant or conflicting. For example, a1=30, b1=30, the correlation judgment R1 can be “intersection.” In this way, the data unit A and the data unit B can be combined, and used for the data processing module 102 to generate the visualization diagram 103.

In some embodiments, the data unit being processed as the project data set is visualized by the data processing module. The project data set defines the data attributes by the operating platform, which the data attributes are processed and generated by the data processing module. In this way, by using the correlation judgment R1 to judge the relationship of the data values between each other, the data processing module can be used to copy, move, or delete the data values of the data unit and the data values of the project data set.

Next please refer to FIG. 11, which is a block diagram I of the sixth embodiment of the present disclosure. This embodiment mainly provides an embodiment of a data processing module 202 labeling the data unit A. The data processing module 202 further includes a labelled data expansion module 2022. The system for data process 20 further includes a labelled database 2025, an unlabelled database 2026, an automatic data labeling module 2021 signally connected to the labelled database 2025, and an operating platform 201 signally connected to the labelled database 2025, the unlabelled database 2026, and the labelled data expansion module. The labelled data expansion module 2022 can access the unlabelled database 2026 and the labelled database 2025, and includes a labeling pattern data set 2024 being able to store a labeling pattern and an expansion unit 2023 being able to execute a labeling algorithm 2028.

The labelled database 2025 is used to store labelled data units, and the unlabelled database 2026 is used to store unlabelled data units.

The operating platform 201 includes a labeling pattern editing interface 2011 and a data labeling prediction interface 2015, and the labeling pattern editing interface 2011 can be used to input data and perform editing operations to generate at least one confirmation labeling pattern 2013. The operating platform 201 of this embodiment is provided for system users to input data and perform editing, adding, and deleting. In addition, the operating platform 201 can input data and perform editing, adding, and deleting by an application program interface (API). The data labeling prediction interface 2015 can be used to input data and display forecast results. The automatic data labeling module 2021 of this embodiment can be configured to perform data labeling prediction.

The labelled data expansion module 2022 performs operations to generate at least one added labeling data unit 2027 according to at least one confirmation labeling pattern 2013 and unlabelled database 2026, and stores the at least one added labeling data unit 2027 in the labelled data database 2025 in order to expand the labelled database 2025. The data processing module 202 is used to process the added labeling data unit 2027 and generate a visualization diagram 203.

Please further refer to FIG. 12, which is a flowchart of a method for data labeling according to the sixth embodiment of the present disclosure, and is applicable to the system for data process 20 shown in FIG. 10. The steps of the method for data labeling of the sixth embodiment are described as follows: performing step S1, the labeling pattern editing interface 2011 of the operating platform 201 receives the data unit A or perform editing operation, and perform step S21, the labelling pattern editing interface 2011 receives at least one labeling pattern. Then, performing step S22, the labeling pattern editing interface 2011 configure the received at least one labeling pattern as at least one confirmation labeling pattern 2013. In this embodiment, the system user performs editing on the labeling pattern editing interface 2011 to input the labeling pattern, and the labeling pattern editing interface 2011 uses the labeling pattern inputted by the system user as the confirmation labeling pattern 2013. It is supplemented that, in other embodiments, the labeling pattern editing interface 2011 may also receive the input of data unit A or perform editing, adding, and deleting via an external application program interface.

After obtaining the at least one confirmation labeling pattern 2013, perform step S23, and the labelled data expansion module 2022 stores the at least one confirmation labeling pattern 2013 in a labeling pattern data set 2024. In this embodiment, labelled data expansion module 2022 first performs a test according to the at least one confirmation labeling pattern 2013 and the labeling pattern in the labeling pattern data set 2024; then, after the test is confirmed, the at least one confirmation labeling pattern 2013 is stored and updated to the labeling pattern data set 2024. The labelled data expansion module 2022 tests whether there is a repetition or a conflict between the at least one confirmation labeling pattern 2013 and the data unit in the labeling pattern data set 2024. In this way, the repetition or the conflict between the labeling patterns can be removed. In other embodiments, the expansion unit 2023 may also perform the aforementioned test.

Then perform step S31, the expansion unit 2023 of the labelled data expansion module 2022 executes labeling algorithm 2028 according to the labelling pattern data set 2024 and an unlabelled database 2026 to generate at least one added labeling data unit 2027; and perform step S4 to store the at least one added labeling data unit 2027 in a labelled database 2025. Specifically, in step S31 of this embodiment, the expansion unit 2023 is based on the at least one confirmation labeling pattern 2013 in the labeling pattern data set 2024 and the labeling patterns that has been stored in the labeling pattern data set 2024, to execute the labeling algorithm 2028 on the data unit in the unlabelled database 2026 in order to label the data unit in the unlabelled database 2026, and the at least one added labeling data unit 2027 is generated. After performing step S31, the expansion unit 2023 stores the generated added labeling data unit 2027 in the labelled database 2025 to expand the labelled database 2025. The labeling algorithm 2028 of this embodiment may be a string searching algorithm or a maximum matching algorithm.

In the case that there is no pre-existing data unit in the labeling pattern data set 2024, step S23 of this embodiment may optionally not be performed. In this case, in step S31, the expansion unit 2023 labels the data unit in the unlabelled data base 2026 according to the at least one confirmation labeling pattern 2013 generated in the step S22.

The method for labeling data unit of the data processing module 202 in this embodiment can further perform step S51, that the data labeling prediction interface 2015 of the operating platform 201 receives an unlabelled data unit; and step S52 is performed, that an automatic data labeling module 2021 performs data labeling prediction on the unlabelled data according to the labelled database 2025, and transmits the prediction result corresponding to the unlabelled data to the operating platform 201. The automatic data labeling module 2021 of this embodiment may execute algorithms such as Recurrent Neural Network, Conditional Random Field, and Maximum-Entropy Markov Model. Then, the operating platform 201 displays the prediction result corresponding to the unlabelled data.

The system for data process 20 shown in FIG. 11 and FIG. 12 can be used to process corpus data, image data, or audio data. In the case of processing corpus data, the unlabelled database 2026 is a corpus database, and the labelled database 2025 is a labelled corpus database, that is, the labelled corpus data is stored. In addition, the unlabelled data is corpus data. The confirmation labeling pattern is used to label corpus data, which may include at least one of morphological information, syntax information, and semantic information.

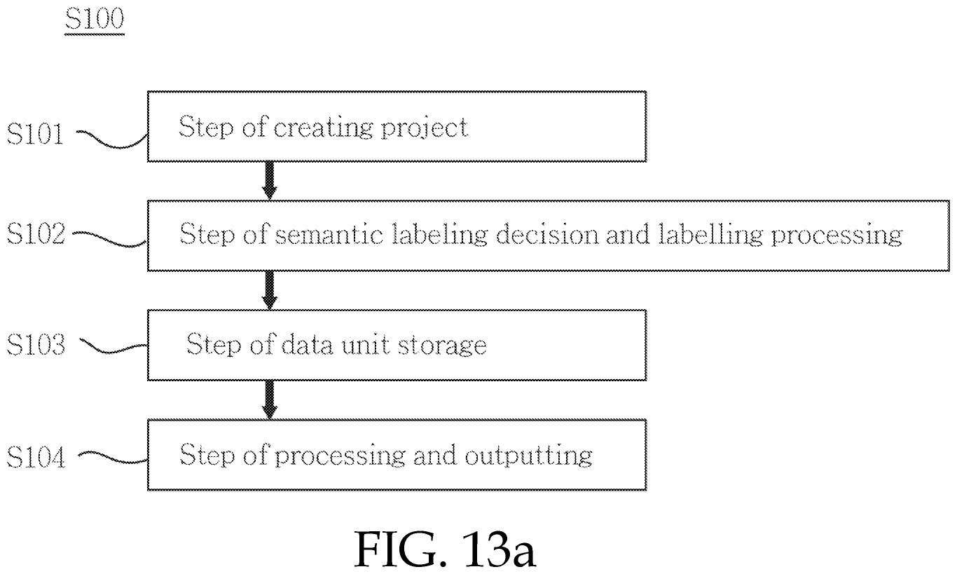

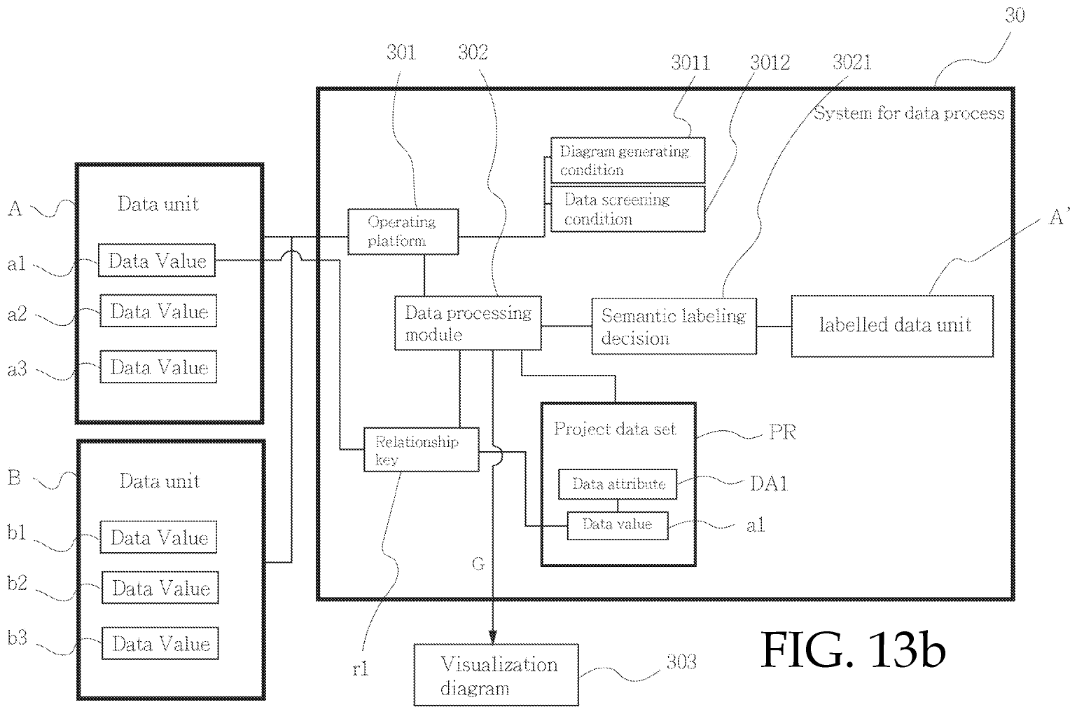

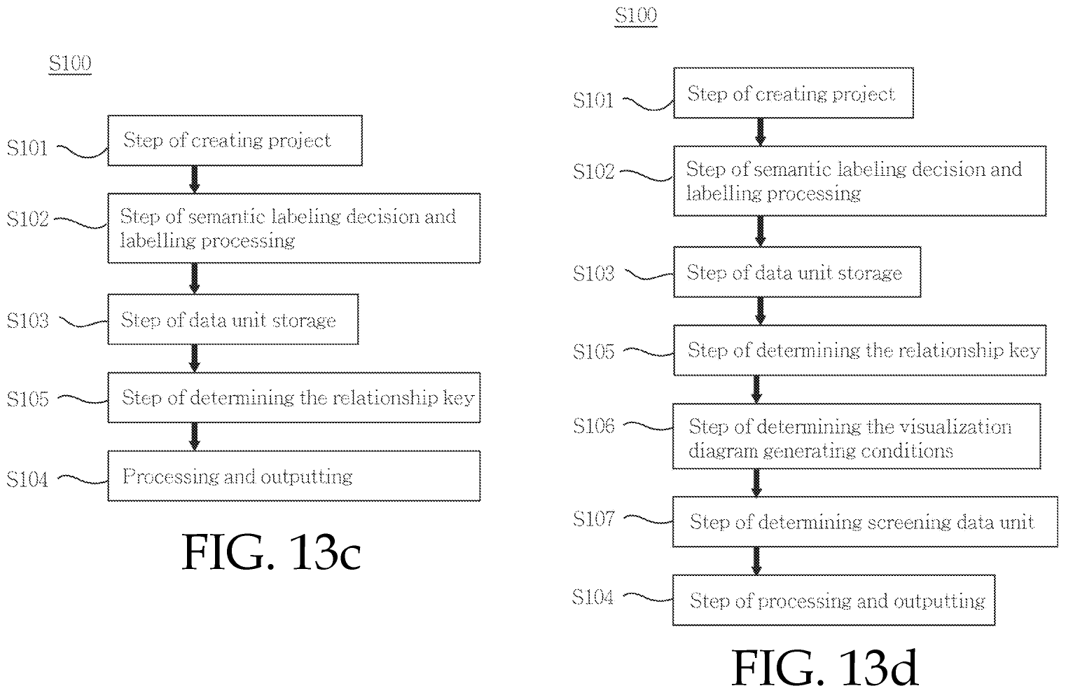

Next, please refer to FIG. 13a and FIG. 13b for the block diagram and flowchart of a system for data process 30 and a method for data process S100 of the present disclosure. The steps are described as follows: perform step S101 of creating project. A project data set PR is generated by the operating platform 101 to access at least one data unit A including a plural of data values a1-a3. The project data set PR includes at least one data attribute DA1.

Perform step S102 of semantic labeling decision and labeling processing. The data processing module 102 processes at least one data unit A and determines a semantic labeling decision 3021, and outputs a labelled data unit A′. The semantic labeling decision 3021 makes one of the plural data values a1-a3 correspond to at least one data attribute DA1. When the at least one data unit A is in an unstructured data format, the data processing module 302 performs an automatic semantic labeling step to complete the semantic labeling decision 3021 so that the data value a1 corresponds to the data attribute DA1. Perform step S103 of data unit storage. The data processing module 302 stores the labelled data unit A′ to the project data set PR. Perform step S104 of processing and outputting. The data processing module 302 processes the project data set PR to generate a visualization diagram 303.

Please refer to FIG. 13c for a variation of the method for data process S100. The method for data process S100, wherein the system for data process 30 further includes a plurality of data units A and data units B. Before step S104 is performed, it includes step S105 of determining the relationship key: the data processing module 302 determines a relationship key r1. The relationship key r1 determines that the data value a1 corresponds to the data attribute DA1 of the project data set PR.

Please refer to FIG. 13d for another variation of the method for data process S100. The method for data process S100 further includes the step S106 of determining the visualization diagram generating conditions: the operation platform is connected to the data processing module, the operation platform 301 determines a diagram generating condition, and the data processing module 302 performs step S104 after screening the project data set according to the diagram generating condition.

Please refer to FIG. 13d again, the method for data process may further include step S107 of determining screening data unit: the operating platform 301 determines a data screening condition 3012, and the data processing module 302 screens the data unit A according to the data screening condition 3012, and then performs step S103 and step S104.

The above are only preferred embodiments of the present invention, and are not used to limit the scope of embodiment of the present invention; anyone who is familiar with this technique and makes equal changes and modifications without departing from the spirit and scope of the present invention shall be covered by the claims of this disclosure.

To sum up, the present disclosure obtains the patent requirements “utility,” “novelty” and “non-obviousness”; the applicant filed an application for a patent before the Patent Office in accordance with the provisions of the Patent Law.

1. A system for data process, comprising:
an operating platform for storing and reading a data unit;
a data processing module, signally connected to the operating platform; and
the data unit is structured or unstructured;
wherein the data processing module labels and processes the data unit, and generates a visualization diagram.

2. The system of claim 1, further comprises:
an unstructured database;
a structured database;
the operating platform signally connected to the unstructured database and the structured database;
wherein at least one of the unstructured database and the structured database storing at least one of the data unit; and
the data processing module labeling and processing at least one of the data unit, and generating the visualization diagram.

3. The system of claim 2, wherein the unstructured database stores at least one of the data unit, and the structured database stores at least one another data unit.

4. The system of claim 2, wherein the data processing module further used for automatically labeling the data unit; and
when the data unit is unstructured, the data processing module automatically labeling and processing the data unit;
wherein the data processing module determines whether to automatically label the at least one data unit according to which of the database the at least one data unit belongs:
when the at least one data unit is stored in the unstructured database, the data processing module automatically labels and processes the at least one data unit, and generates the visualization diagram; and
when the at least one data unit is stored in the structured database, the data processing module processes and generates the visualization diagram.

5. The system of claim 1, wherein the data processing module is further used to process the at least one data unit and generates a visualizable data set;
wherein the data processing module generates the visualization diagram according to the visualizable data set.

6. The system of claim 5, wherein the operating platform further used to import the visualizable data set, which is generated by the data processing module pre-processed the at least one data unit; the data processing module generates the visualization diagram according to the visualizable data set being imported.

7. The system of claim 5, further comprising a visualizing module signally connect to the operating platform and the data processing module; wherein the visualizing module generates the visualization diagram according to the visualizable data set.

8. The system of claim 2, wherein the at least one data unit comprises unstructured data, structured data, semi-structured data, or a combination thereof.

9. The system of claim 8, wherein the structured data includes structured patient data, medication record, drug data, medicine data (pharmaceutical raw materials), doctor's advice data, doctor's data, equipment data, department data, hospitalization data, examination data, shift report, or a combination thereof.

10. The system of claim 8, wherein the unstructured data includes unstructured texts, case history, doctor's advice, patient data, medication record, medicine profile data, drug profile data, doctor's data, equipment data, department data, hospitalization data, examination data, shift report, or a combination thereof.

11. The system of claim 1, the operating platform comprises a visualization diagram editing interface used to configure the generating conditions of the visualization diagram and decide a diagram generating condition;
wherein the data processing module processes the at least one data unit and generates the visualization diagram according to the diagram generating condition.

12. The system of claim 11, the diagram generating condition comprises displayed field condition, data screening condition, diagram type, or a combination thereof.

13. The system of claim 11, wherein the data unit comprises a data value, corresponding to a data attribute of the data unit; and
the diagram generating condition decide the visualization diagram displays at least one of the data attribute.

14. The system of claim 11, wherein the diagram generating condition comprises: a data screening condition, having: data set condition, classification and clustering condition, data attribute condition, arithmetic unit condition, tagged value condition, or any combination thereof, wherein the data processing module screens the data value according to the diagram generating condition, and processes the data value after being screened to generate the visualization diagram.

15. The system of claim 11, wherein the diagram generating condition comprises: a diagram category, having: data list, data table, basic frequency table, percentage table, co-occurrence matrix, co-occurrence list, population distribution, bar graph, line graph, table, pie graph, histogram, statistical graph, scatter graph, bubble graph, surface graph, radar graph, horizontal bar graph, timeline, organ/body tissue chart, or a combination thereof.

16. The system of claim 11, further comprises: a historical data module signally connected to the data processing module and the operating platform for storing the diagram generating condition, the visualization diagram, or any combination thereof.

17. The system of claim 1, wherein the at least one data unit comprises at least one data value;
wherein the operating platform is used to generate a project data set, including at least one project data attribute;
wherein the operating platform determines that the at least one project data attribute corresponds to the data attribute, which the at least one data value belongs to; and
the data processing module generates the visualization diagram according to the project data set.

18. The system of claim 17, wherein the project data set comprises:
a plurality of project data attributes, and the at least one data unit including a plurality of data values;
wherein the operating platform determines:
one of the plurality of project data attributes corresponds to the data attribute one or more than one of the plurality data values belong to; or more than one of the plurality of data attributes correspond to the data attribute one or more than one of the plurality of data values belong to.

19. The system of claim 1, further comprises a plurality of data units, and any one of the plurality of the data units includes at least one data value;
wherein the operating platform is further used to determine at least one relationship key;
wherein the relationship key corresponds to one of the plurality of data units and another one of the plurality of data units; and
wherein the data processing module further generates the visualization diagram according to the at least one relationship key.

20. The system of claim 19, wherein the at least one relationship key corresponds to the data attribute belonging to the at least one data value of one of the plurality of data units and the data attribute belonging to the at least one data value of the other of the plurality of data units;
wherein data processing module processes the plurality of data values and generates the visualization diagram.

21. The system of claim 1, wherein the data unit comprises a plurality of data values, and the data processing module determines at least two of the plurality of data values at least one relevance determination, wherein the relevance determination comprises “greater than”, “equal to”, “less than”, “not equal to”, “greater than or equal to”, “including”, or “less than or equal to”; the data processing module further generates the visualization diagram according to the relevance determination.

22. The system of claim 21, wherein when the at least two of the plurality of data values are corpus data, the relevance determination includes a logical operation judgment, a similar semantic judgment, or a combination thereof.

23. The system of claim 1, wherein the data unit comprises: a patient data set, including a plurality of data values; the data process module processes the patient data set to generate the visualization diagram;
wherein any one of the plurality of data values corresponds to at least one data attribute, the data processing module generates the visualization diagram according to the plurality of data values and the data attribute;
wherein the visualization comprises representations of the at least one data attribute.

24. The system of claim 23, wherein the data attribute comprises a plurality of time data, a first data attribute, and a second data attribute;
one of the plurality of data values corresponds to the first data attribute and one of the plurality of time data;
another one of the plurality of data values corresponds to the second attribute and the one of the plurality of time data;
wherein the data processing module generates a co-occurrence analysis result according to the one of the plurality of time data, the one of the plurality of data values, and the another one of the plurality of data values;
the visualization diagram comprises the co-occurrence analysis result.

25. The system of claim 24, comprises an analysis module signally connected to the operating platform; the analysis module used to process the co-occurrence analysis result, predict the patient data set, and generate a predicted result.

26. The system of claim 24, the co-occurrence result further comprising
statistical analysis result, probability analysis result, or a combination thereof.

27. The system of claim 26, the plurality of time data comprises a time record of patient's examination, a time record of patient's examination report being made, a time record of patient's visit a doctor, or a combination thereof.

28. The system of claim 1, further comprising: a labeling database and an unlabelling database;
wherein the data processing module comprises a labelled data expansion module;
the operating platform signally connected to the labeling database, the unlabeling database and the labelled data expansion module, and comprised a labeling pattern editing interface, and the labeling pattern editing interface used for inputting data units and performing editing operation to generates at least one confirmation labeling pattern;
wherein the labelled data expansion module performs operation to generates at least one added labeling data unit according to the at least one confirmation labeling pattern and the unlabeling database, and restores the at least one added labeling data unit to the labeling database;
wherein the data processing module used for processing the added labelled data unit, and generating a visualization diagram.

29. A method for data process, a data processing module is used to process one of unstructured data and structured data, to process at least one data unit, and perform the following steps:
(A) a step for project creation: an operating platform generates a project data set for accessing the at least one data unit including a plurality of data values; the project data set includes at least one data attribute;
(B) a step for semantic labeling decision and labeling process: the data processing module processes the at least one data unit, determines a semantic labeling decision, and outputs a labelled data unit, the semantic labeling decision makes one of the plurality of data values to correspond to the at least one data attribute;
when the at least one data unit is unstructured data format, the data processing module performs a step for automatic semantic labeling to complete the semantic labeling decision makes one of the plurality of data values to correspond to the at least one data attribute;
(C) a step for data unit storage: the data processing module stores the labelled data unit to the project data set; and
(D) a step for process and output: the data processing module processes the project data set to generate a visualization diagram.

30. The method of claim 29, further comprises a plurality of data units, wherein before the step of (D) is executed, the method comprises:
(E) a step for determining a relationship key: the data processing module determines a relationship key, and the relationship key determines one of the plurality of data values of one of the plurality of data units corresponding to one of the at least one data attribute of the project data set.

31. The method of claim 29, further comprises:
(F) determining visualization diagram generating condition, an operating platform is connected to the data processing module, the operating platform determines a diagram generation condition, and after the data processing module screening the project data set according to the diagram generation condition, executes the step of (D).

32. The method of claim 29, further comprises:
(F) a step for screening data units: the operating platform determines a data screening condition, the data processing module performs step (C) and step (D) after screening the data unit according to the data screening condition.
